(19) United States
(12) Patent Application Publication
(10) Pub. No.: US 2004/0058992 A1
(43) Pub. Date: Mar. 25, 2004

(54) Apparatus for the introduction of a new system for the treatment of maxillary and frontal sinusitis and neuritis and neuralgia of the trigeminal nerve

(76) Inventors: Andrea Marinello, Verona (IT); Vittoria Marinello, Verona (IT)

Correspondence Address: FROMMER LAWRENCE & HAUG, 745 FIFTH AVENUE- 10TH FL., NEW YORK, NY, 10151, US

(21) Appl. No.: 10/662,775

(22) Filed: Sep. 15, 2003

(30) Foreign Application Priority Data
Sep. 25, 2002 (IT) VR2002A000094

(51) Int. Cl. A61K 31/26; A61K 9/70

(52) U.S. Cl. 514/514; 424/443

(57) ABSTRACT

The invention relates to a device and method for the treatment of illnesses of paranasal sinuses, and neuritis and neuralgia of the trigeminal nerve. More particularly, the present invention provides for a device and method for treating sinusitis. Even more particularly, the present invention provides for a device and method for administering a revulsive having an active ingredient to treat inflammation and infection. The device comprising a facial mask, an inflatable elastic pad affixed to the mask and a compress containing a revulsive having an active ingredient. In addition, a method of administering a revulsive to a portion of a person's skin in need thereof is disclosed. Other improvements and a method for implanting the device are disclosed as well.

### CROSS-REFERENCE TO RELATED APPLICATION

This application claims priority benefits from Italian Patent Application No. VR2002A000094 filed Sep. 25, 2002, the disclosure of which is incorporated herein by reference.

### FIELD OF THE INVENTION

The present invention relates generally to the field of treatment of illnesses of the paranasal sinuses, and neuritis and neuralgia of the trigeminal nerve. More particularly, the present invention provides for a device and method for treating sinusitis. Even more particularly, the present invention provides for a device and method for administering allyl isothiocyanate to bones underlying the mucous membranes in the sinuses to treat inflammation and infection.

### BACKGROUND OF THE INVENTION

Acute and chronic sinusitis are illnesses of the paranasal sinuses, cavities that are connected to nasal cavities by means of canals. There are four paranasal sinuses: the maxillary, frontal, sphenoid and ethmoid. These sinuses are covered in a mucous that is a continuation of the nasal cavity mucous and susceptible to the same alterations.

Sinuses are affected by simple colds. Sinusitis occurs when the symptoms relating to sinusoid cavities become independent from the nasal cavities. Sinusitis is an inflammation of the paranasal sinuses, and specifically, the frontal sinuses. The inflammation may derive from a nasal or a dental infection, or an infection carried through the bloodstream. The presence of predisposing factors, such as excessively narrow nasal passages, deviation of the septum, etc. favor sinusitis.

Sinusitis can be acute or chronic. The acute form is an inflammation of one or two sinuses, originating from a nasal infection but may develop into an acute purulent sinusitis or even ulcerous-necrotic sinusitis. Some acute sinusitis can become chronic if the inflammations or lesions are not limited to the sinus mucous but spread to the bone tissue. There are two varieties of chronic sinusitis, local or general. The types of chronic sinusitis depend on the extent of inflammations or lesions, and the seriousness of the symptoms, such as purulent sinusitis, hyperplasia, etc.

A characteristic of the more serious forms of chronic sinusitis is obstruction of nasal respiration, a serious disturbance that can have long term repercussions on the functioning of the respiratory tract. Bronchial asthma, headaches, migraines, neuritis, and neuralgia of the first or second opthalmic or maxillary branches of the trigeminal nerve can often be ascribable to effects of chronic sinusitis. Sinusitis results in swelling of the mucous membranes lining the nasal and sinus passageways. As the swelling blocks the narrow openings from the nose into the sinuses, the sinuses are unable to drain, and mucous and debris from the lining of the sinuses build up, stabilize and eventually impregnate the passage walls to the bone, thus reducing respiratory capacity. As the sinus, affected by sinusitis, fills with exudate, the exudate hardens and becomes less transparent. An x-ray of healthy paranasal sinuses shows a sharp and clear image, while for those affected by chronic sinusitis the x-ray image is opaque, and in the more serious cases the x-ray image shows sclerotic sinuses.

From the above, it should be evident that chronic sinusitis is among the internal inflammations or lesions that pose grave consequences for human well-being. Proper respiration is vital for the oxygenation of tissues. For example, an individual can survive for days without food but faces certain death if unable to breathe for a few minutes. Given the importance of the problem, medical science has been slow to find a treatment for this disease. The first treatments of sinusitis were antibiotics combined with local pharmaceuticals to keep the sinusoidal cavities open; local drugs with anti-bacterial and anti-inflammatory action; thermal aerosol equipment; and inhalation techniques. However, these treatments are inadequate and persisting symptoms force surgical operations: opening the infected sinus, emptying its contents and scraping the sinus walls down to the bone. It must be said, however, that prolonged chronic sinusitis proved for the most part resistant to every treatment. Entrenched in loco and reducing respiratory capacity, sinusitis constitutes a risk to a patient who may be affected by the addition of other ailments accompanying it. As mentioned earlier, until now treatments included thermal applications and inhalant for sinusitis, given the fact that heat administered through compresses, hydrotherapy, mud, steam and electricity on a part of the body provokes a dilation of the blood vessels and a consequent increase in the flow of blood. But while medicine has been able, by these means, to provoke these vasomotory reactions in superficial vessels, it has been unable to produce similar reactions on internal and deeper vessels. By and large, these treatments have helped with superficial ailments but have done little to help chronic sinusitis where damage has penetrated and affected the bony walls of the sinuses. This is all due to a deep-rooted belief that inhalant treatments, muds, compresses, hydrotherapies, etc. had the power to provoke reactions on superficial blood vessels only and not on internal and deeper blood vessels.

Consequently, a need exists in the art for a method and device for treating sinusitis. The present lack of effective therapies for maxillary, frontal and chronic sinusitis in its various forms can be remedied by the method and device of the present invention. In contrast to the prior art, the present invention provides for a facial mask with a compression mechanism that acts on a compress impregnated with an active ingredient, i.e. allyl isothiocyanate, and promotes the absorption of the active ingredient into the skin to reach bones underlying the mucous membranes in the sinuses to treat inflammation and infection.

The new finding rests on the assumption that along with heat administered using mud, steam, electricity, etc. on a part of the body, there are other substances, known as revulsives that produce analogous effects; among these revulsives are cresses, including mustard, radish, etc. The active ingredient in these substances (allyl isothiocyanate [C₃H₅NCS]) causes a vasomotory reaction. This active ingredient comes in different concentrations in various cresses and the difference in concentration makes it impossible to administer safely as it provokes blistering of the skin. However, the Applicants have found that using cresses in a natural state can result in avoiding their harmful effects. When the cresses are used in a dried form in a compress and soaked with water in its returned state at 10° C., the active ingredient is diluted and maintains its vasomotory properties while losing its harmful effects.

In contrast to the prior art, the present invention provides a method and device to administer a revulsive or cress, and specifically the active ingredient, allyl isothiocyanate, to promote the absorption of the active ingredient to bones underlying the mucous membranes in the sinuses to reduce inflammation and infection without harmful side effects. The device comprises a facial mask with a compression mechanism and a compress impregnated with a revulsive or a cress having an active ingredient to promote the absorption of the active ingredient into the skin until it reaches the inflammation or the damaged area and the bones underlying the mucous membranes.

### OBJECTS OF THE INVENTION

Therefore, it is an object of the invention to provide a device and method for treating sinusitis.

It is an objective of the invention to provide a device and method for administering an active ingredient contained in revulsives, and specifically cresses, to treat illnesses of paranasal sinuses and neuritis and neuralgia of the trigeminal nerve.

It is still another objective of the invention to provide a device adapted to administer allyl isothiocyanate to treat inflammations and specifically bones underlying the mucous membranes in the sinuses.

It is still another objective of the invention to provide a uniform packaged compress method for using revulsives or cresses to treat illnesses of sinusitis.

Various other objects, advantages and features of the present invention will become readily apparent from the ensuing detailed description.

### SUMMARY OF THE INVENTION

Accordingly, a device is provided comprising a facial mask, an inflatable elastic pad affixed to the mask and a compress comprising a revulsive having an active ingredient.

A device is also provided comprising a facial mask, an inflatable elastic pad affixed to the facial mask and a compress comprising a revulsive having an active ingredient, wherein the device promotes the absorption of the active ingredient to bones underlying mucous membranes in the sinus of a person in need thereof

A method of treating sinusitis is provided comprising the step of securing a device to the head of a person, the device comprising a facial mask, an inflatable elastic pad affixed to the facial mask and a compress comprising a revulsive having an active ingredient.

A method of using a revulsive having an active ingredient to treat inflammation is also provided comprising the steps of: providing a revulsive in a dried form; soaking the revulsive with water at 10° C.; and squeezing the compress to force out the active ingredient onto a portion of a person's skin to reduce inflammation.

These and other embodiments of the invention are provided in, or are obvious from, the following detailed description of the invention.

### BRIEF DESCRIPTION OF THE DRAWINGS

The following detailed description given by way of example, but not intended to limit the invention solely to the specific embodiments described, may best be understood in conjunction with the accompanying drawings in which:

FIG. 1 is a facial mask of the first embodiment of the present invention.

FIG. 2 is a facial mask of the second embodiment of the present invention.

FIG. 3 is a facial mask having bands of the first embodiment of the present invention.

FIG. 4 is a facial mask having bands of the second embodiment of the present invention.

FIG. 5 is an inflatable elastic pad of the first embodiment of the present invention.

FIG. 6 is an inflatable elastic pad of the second embodiment of the present invention.

FIG. 7 is an inflatable elastic pad affixed to the facial mask of the first embodiment of the present invention.

FIG. 8 is an inflatable elastic pad affixed to the facial mask of the second embodiment of the present invention.

FIG. 9 is a rubber pump.

FIG. 10 is a compress of the first embodiment of the present invention.

FIG. 11 is a compress of the second embodiment of the present invention.

FIG. 12 is an exploded view of the first and second embodiments of present invention.

### DETAILED DESCRIPTION

The present invention provides for a device and method for treating sinusitis. The device of the present invention is designed to promote the absorption of the active ingredient contained in revulsives and cresses into the skin of a person to reach bones underlying the mucous membranes in the sinuses to treat inflammation and infection. The device comprises a mask, an inflatable elastic pad affixed to the facial mask, and a compress comprising a revulsive or a cress having an active ingredient.

The mask of the present invention is made of any material suitable in the art, for example, plastic, and is resistant to deformation. The mask may be made in a variety of sizes to meet different persons' needs. The mask can be fitted for the upper part of the face or the lower part of the face. In addition, the mask may include openings for a person's eyes. Further, the mask allows the nostrils and mouth of the wearer to freely accommodate respiratory functions. Furthermore, the mask is adapted to be secured to the head of the wearer to firmly hold the mask in place. For example, two bands of strong fibers secured behind the neck holds the mask against the lower or upper part of the face. However, a skilled artisan would readily understand other methods of securing the mask to the head of the wearer.

The device of the present invention incorporates an inflatable elastic pad having two membranes that have substantially the same shape as the mask. The two membranes form a compression chamber. The inflatable elastic pad is affixed to the edges of the mask. A skilled artisan would readily understand other methods of securing the inflatable elastic pad to the mask. In addition, the elastic pad is inflatable via a small rubber tube that is inserted in the membranes and runs to a small rubber pump with a valve, which pumps air into the compression chamber. However, a skilled artisan would readily understand other methods for inflating the pad.

Further, the compress comprises a revulsive or cress containing an active ingredient, such as allyl isothiocyanate (C₃H₅NCS). This compress has substantially the same shape as the elastic pad and is soaked in water at 10° C. with the revulsive or cress enclosed therein. The compress is designed so that when pressure is applied, the active ingredient may be forced out of the compress. However, a skilled artisan would readily understand other methods of forcing the active ingredient out of the compress. The active ingredient is diluted and may be delivered to the skin without harmful side effects. The compress is made of any material suitable in the art, for example, cloth or mesh.

The embodiments of the present invention can be used for the treatment of sinusitis and more particularly, the present invention provides for a device and method for promoting the absorption of an active ingredient to bones underlying the mucous membranes in the sinuses to reduce inflammation and infection.

The device described above has a number of functions. One function is static, that is, through the action of the two bands of strong fibers held behind the neck holding the compress against the lower or upper part of the face. Another is dynamic, that is, inflating the compression chamber using the pump and provoking the following effects:

a) squeezing the compress to force out the active ingredient (allyl isothiocyanate);

b) opening skin pores to which pressure is applied; and

c) forcing the active ingredient through the opened skin pores in the direction of the damaged or inflamed area.

The simultaneous combined action of these factors results in the active ingredient, e.g., allyl isothiocyanate, entering the pores. It is readily understood that other active ingredients, and combinations of two or more active ingredients, can be used as well.

A portion of the medication is dispersed in the blood stream and a portion reaches the vessels feeding the injured area, thus provoking enlargement, greater supply of blood and consequent treatment effects. The effects take the form of mucous becoming fluid.

Given that the active ingredient must penetrate the bone that is impregnated with mucous, the pressure applied must be appropriate. Because the resulting fluid must escape via the nasal passages, it is advisable that the present methods are applied in combination with traditional treatments, which ease the flow of mucous through the nasal passages. The invention is not intended to be limited purely to the application described above.

Referring now to the drawings, in which like reference numerals are used to refer to the same or similar elements, the device comprises a facial mask, an inflatable elastic pad affixed to the facial mask and a compress containing an active ingredient.

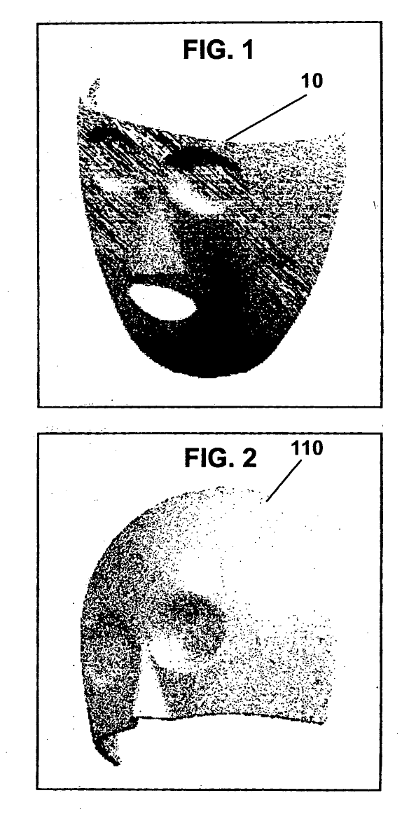

FIG. 1 describes the facial mask of the first embodiment of the present invention. The facial mask (10) is applied to the skin of the lower part of the face for treating the maxillary sinusitis. FIG. 2 describes the facial mask of a second embodiment of the present invention. In the second embodiment, the facial mask (110) is applied to the skin of the upper half of the face for to treat frontal and neuritic sinusitis. In both cases the mask provides openings for a person's eyes. In addition, in both cases a person's nostrils and mouth are free to accommodate respiratory functions. The mask is made of any suitable material in the art, for example plastic, and resistant to deformation.

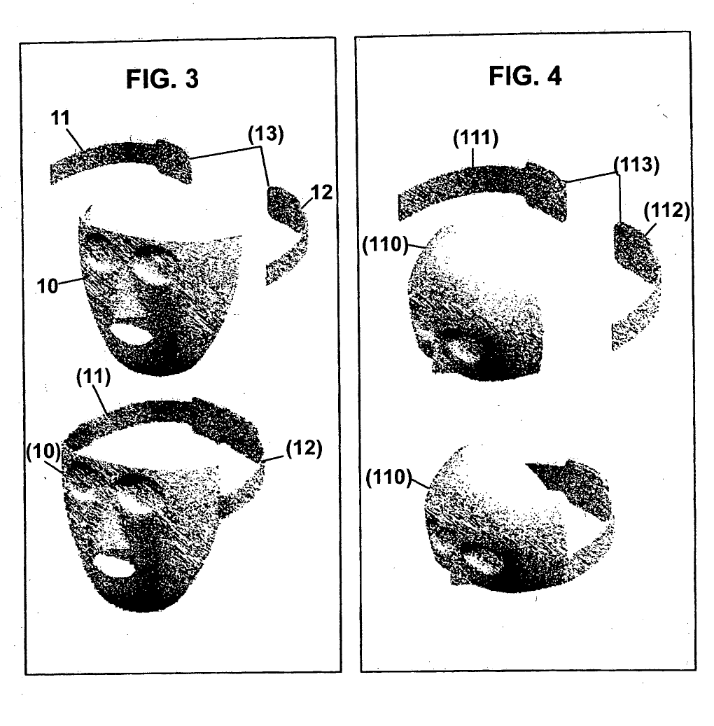

FIG. 3 illustrates the mask of the first embodiment of the present invention having bands to secure the facial mask to the lower face of the wearer. There are two holes (not shown) on each side of the facial mask (10) to which are affixed two bands of strong fiber (11) and (12) which go around the neck (not shown) these bands have a hook and loop fastener (13) at the end of the bands, or other means of securing the mask to firmly stay in place, such as Velcro, etc.

FIG. 4 illustrates the mask of the second embodiment having bands to secure the facial mask to the upper face of the wearer. There are two holes (not shown) on each side of the facial mask (110) to which are affixed two bands of strong fiber (111) and (112) which go around the neck (not shown) these bands have a hook and loop fastener (113) to secure the mask, or other means of securing the mask to firmly stay in place. In either case, the bands join around the back of the neck holding the mask firmly in place.

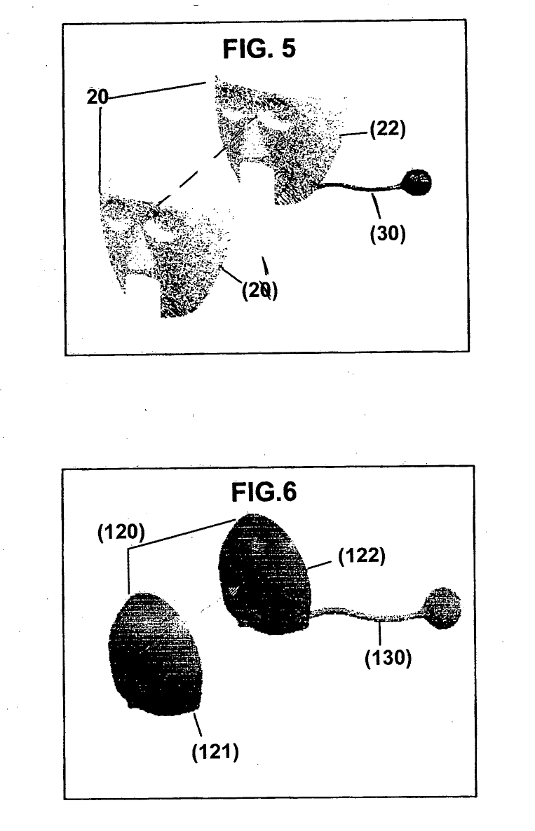

FIG. 5 illustrates an inflatable elastic pad (20) comprising a first membrane (21) and a second membrane (22) of elastic material that have substantially the same shape as the facial mask (10) of the first embodiment. The elastic pad (20) is affixed to the edges of the facial mask (10) and inflatable via a small rubber tube (30). First membrane (21) and second membrane (22) form a compression chamber as described by the dashed lines.

FIG. 6 illustrates an inflatable elastic pad (120) comprising a first membrane (121) and a second membrane (122) of elastic material that have substantially the same shape as the facial mask (110) of the first embodiment. The elastic pad (120) is affixed to the edges of the facial mask (110) and inflatable via a small rubber tube (130). First membrane (121) and second membrane (122) form compression chamber as described by the dashed lines.

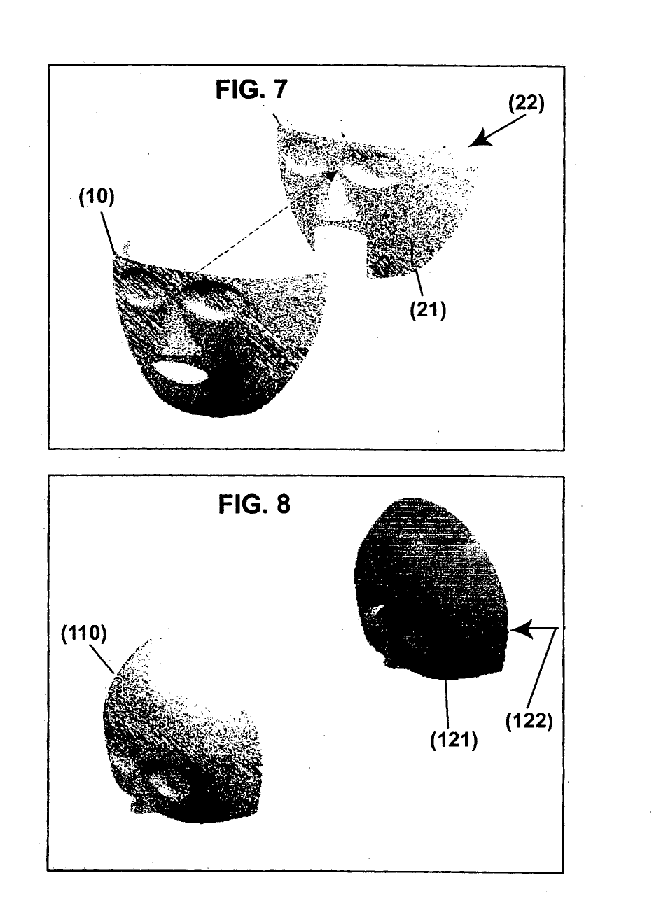

FIG. 7 illustrates that first membrane (21) adheres firmly to the inside of the facial mask (10) of the first embodiment of the present invention.

FIG. 8 illustrates that first membrane (121) adheres firmly to the inside of the facial mask (110) of the second embodiment of the present invention.

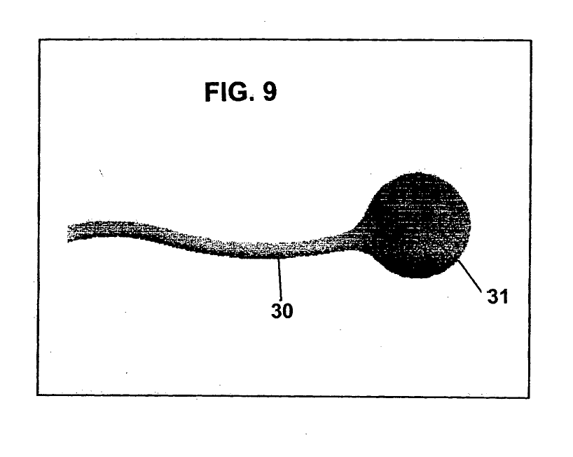

FIG. 9 illustrates rubber tube (30), which is inserted between membrane (21) and second membrane (22) and runs to a small rubber pump (31) with a valve (not shown), which pumps air into the compression chamber.

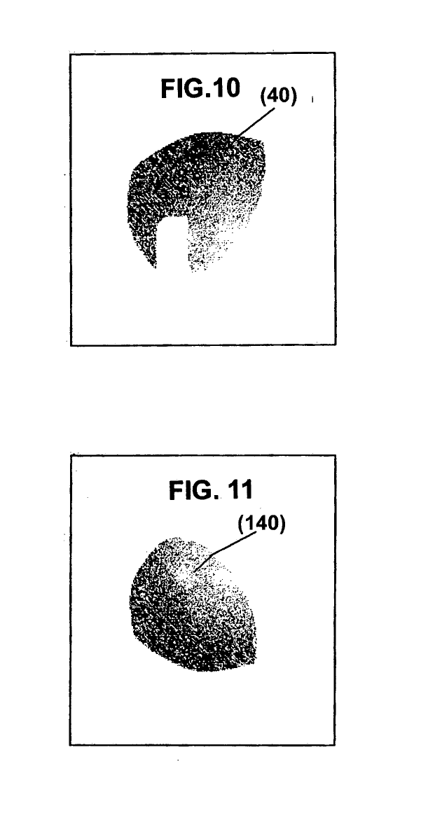

FIG. 10 illustrates compress (40), which contains a revulsive or cress having an active ingredient. Compress (40) has substantially the same shape as elastic pad (20). Compress (40) is soaked in water at 10° C. and is subsequently fitted adjacent to second membrane (22).

FIG. 11 illustrates compress (140), which contains a revulsive or cress having an active ingredient. Compress (140) has substantially the same shape as elastic pad (120). Compress (140) is soaked in water at 10° C. and is subsequently fitted adjacent to second membrane (122).

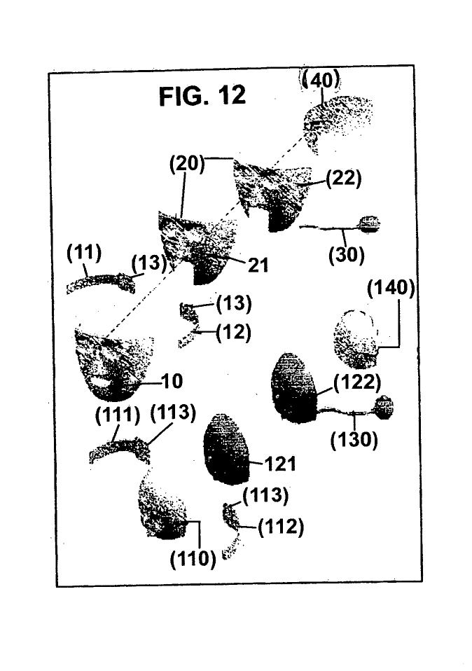

FIG. 12 illustrates an exploded view of the first and second embodiments of present invention. In the first embodiment of the present invention, facial mask (10) having bands (11) and (12) is affixed to first membrane (21). First membrane (21) is attached to second membrane (22) forming a compression chamber. Rubber tube (30) having small rubber pump (31) with a valve, is connected to pump air into the compression chamber. Compress (40) soaked in water at 10° C., containing a revulsive, or a cress comprising an active ingredient such as a allyl isothiocynate, is subsequently fitted adjacent to second membrane (22).

Similarly, in the second embodiment of the present invention, facial mask (110) having bands (111) and (112) is affixed to first membrane (121). First membrane (121) is attached to second membrane (122) forming a compression chamber. Rubber tube (130) having small rubber pump (131) with a valve, is connected to pump air into the compression chamber. Compress (140) soaked in water at 10° C., containing a revulsive or a cress having an active ingredient such as allyl isothiacynate, is subsequently fitted adjacent to second membrane (122).

Although preferred embodiments of the present invention and modifications thereof have been described in detail herein, it is to be understood that this invention is not limited to those precise embodiments and modifications, and that other modifications and variations may be affected by one skilled in the art without departing from the spirit and scope of the invention as defined by the appended claims.

What is claimed is:

1. A device comprising a facial mask, an inflatable elastic pad affixed to the mask and a compress containing a revulsive or cress having an active ingredient.

2. The device according to claim 1, wherein the active ingredient is allyl isothiocyanate.

3. The device according to claim 2, wherein the compress is soaked in water to activate the active ingredient.

4. The device according to claim 3, wherein the water is at a temperature of approximately 10° C.

5. The device according to claim 1, wherein the inflatable elastic pad comprises a first membrane and a second membrane.

6. The device according to claim 5, wherein the first membrane and the second membrane form a compression chamber.

7. The device according to claim 6, wherein a pump is used to inflate the compression chamber.

8. The device according to claim 1, wherein the facial mask comprises at least one opening and means for securing the facial mask to a person's head.

9. The device according to claim 1, wherein the facial mask covers the upper part of a person's head.

10. The device according to claim 1, wherein the facial mask covers the lower part of a person's head.

11. The device according to claim 1, wherein the facial mask is adapted to allow respiration.

12. The device according to claim 1, wherein the facial mask comprises plastic adapted to be resistant to deformation.

13. A device comprising a facial mask, an inflatable elastic pad affixed to the facial mask and a compress comprising a revulsive or cress having an active ingredient, wherein the device promotes the absorption of the active ingredient to bones underlying mucous membranes in a sinus of a person in need thereof.

14. The device according to claim 13, wherein the active ingredient is allyl isothiocyanate.

15. The device according to claim 13, wherein the compress is soaked in water to activate the active ingredient.

16. The device according to claim 15, wherein the water is at a temperature of approximately 10° C.

17. The device according to claim 13, wherein the inflatable elastic pad comprises a first membrane and a second membrane.

18. The device according to claim 17, wherein the first membrane and the second membrane form a compression chamber.

19. The device according to claim 18, wherein a pump is used to inflate the inflatable elastic pad.

20. The device according to claim 13, wherein the facial mask comprises at least one opening and a means for securing the facial mask to a person's head.

21. The device according to claim 13, wherein the facial mask covers an upper part of a person's head.

22. The device according to claim 13, wherein the facial mask covers the lower part of a person's head.

23. The device according to claim 13, wherein the facial mask is adapted to allow respiration.

24. The device according to claim 1.3, wherein the facial mask comprises plastic adapted to be resistant to deformation.

25. A method of treating sinusitis comprising the step of securing a device to a person's head, the device comprising a facial mask, an inflatable elastic pad affixed to the facial mask and a compress comprising a revulsive or cress having an active ingredient.

26. The method according to claim 25, wherein the active ingredient is allyl isothiocyanate.

27. The method according to claim 26, wherein the compress is soaked in water to activate the active ingredient.

28. The method according to claim 27, wherein the water is at a temperature of approximately 10° C.

29. The method according to claim 25, wherein the facial mask is adapted to be secured to a person's head.

30. The method according to claim 25, further comprising the steps of squeezing the compress to force out the active ingredient; opening skin pores to which pressure is applied; and forcing the active ingredient through the opened skin pores of a person in the direction of an inflammation.

31. A method of using a revulsive having an active ingredient to treat inflammation comprising the steps of:
providing a revulsive or cress in a dried form;
soaking the revulsive or cress to activate the active ingredient; and
squeezing the revulsive or cress to force out the active ingredient onto a portion of a person's skin to reduce inflammation.

32. The method according to claim 29, wherein the active ingredient is allyl isothiocyanate.

33. A compress containing an active ingredient in a dried form compressing allyl isothiocyanate which is activated upon the application of water.
